(19) United States
(12) Patent Application Publication
(10) Pub. No.: US 2023/0137136 A1
(43) Pub. Date: May 4, 2023

(54) DEVICES AND METHODS OF TREATING ORAL TISSUES

(71) Applicant: PerioTech, LLC, North Miami, FL (US)

(72) Inventor: Richard JOHNSON, Palm Beach Gardens, FL (US)

(73) Assignee: PerioTech, LLC

(21) Appl. No.: 17/452,874

(22) Filed: Oct. 29, 2021

(51) Int. Cl. A61C 8/02

(52) U.S. Cl. CPC A61C 8/0006; A61F 2002/286

(57) ABSTRACT

A device and method for accelerating the conversion of oral bone graft to alveolar bone following extraction in a patient includes a vibrational dental device having a mouthpiece for contacting the dentition. Instructions are provided for using the vibrational dental device by placing the mouthpiece over the dentition, applying a vibratory force during a predetermined number of sessions throughout a predetermined treatment period, wherein the conversion of bone graft material to mature bone is enhanced and/or accelerated compared to without vibratory treatment.

### TECHNICAL FIELDS

This disclosure concerns treatments of periodontal conditions, more specifically, a method to facilitate periodontic treatments and general oral health using mechanical vibration.

### BACKGROUND

When a tooth is extracted, the extraction socket that held the tooth is filled with blood from the surrounding boney socket walls and soft tissue (e.g., the gums). Hemorrhage due to tooth extraction leads to the formation of a blood clot filling the entire socket. Formation of granulation tissue begins to occur under the influence of the patient's inflammatory response, which further stimulates the recruitment of inflammatory and immune cells. Over time, depending on size, the clot, exposed to the oral environment and other factors, is converted to host bone. Starting from the base of the socket, granulation tissue begins to infiltrate the clot. Epithelial and connective tissue begins to form at the periphery, and the host tissue begins to form new capillaries into the clot from the periphery (i.e., angiogenesis). This process allows the migration of osteoblasts (which form new bone), fibroblasts and other host cells, which further serve to organize and convert the clot to immature osteoid bone such as unmineralized spicules that will over time become more organized and denser bone through mineralization and increased epithelialization. Frequently, bone graft materials are placed into an extraction or excision site to increase bone volume intraorally. The site may be an extraction socket or a site from other surgical procedures resulting in a void or defect in the bone, or the removal of damaged or diseased bone, trauma, or other endodontic or periodontal condition. This is typically the case where, after extraction of a tooth, the volume of a defect in need of repair is larger, or where according to a clinical plan, quickly filling the defect is needed in order to place an implant or other prosthodontic device at the site.

With regards to osseous grafting, the graft material can be an autograft, allograft, synthetic or even a xenograft. The graft material acts as a scaffold maintaining the defect or void volume longer than would be observed with just a clot alone. The peripheral tissue may bleed into the graft material, forming a clot around it, stabilizing the graft material to help contain it in the site and to help introduce host cells into the mixture. Some practitioners are using blood drawn from the patient and processed into autogenous blood concentrates, such as Platelet Rich Plasma (PRP) or Platelet Rich Fibrin (PRF), and mixed with the graft particles to form a stable mass, which is then packed into site. Autogenous blood concentrates have, as the name implies, a concentration of those stimulatory cells from the host, and have been shown to clinically improve bone grafting outcomes. The oral grafting process is often accompanied by pain and inflammation.

Unlike natural teeth, implants have no native periodontal ligament (PDL) between the implant and the bone to which the implant is anchored. As a result, further bone loss and receding of the PDL can result in widening gaps post extraction and result in unwanted mobility of implanted prosthetic teeth. Mobility of an implant is an indication of failure, and without intervention could lead to loss of osseointegration between the implant and adjacent bone. Effective treatments have been demonstrated to be able to treat bone loss around implants when no mobility is present. When mobility already presents with an implant, however, no documented treatment has been proven effective. Therefore, it is desired to have faster high-quality grafting around an implant area after the implant is planted.

Another recognized problem with implanted prosthetic teeth is that over time, inflammation is commonly found around dental implants, a condition known as peri-implantitis. Peri-implantitis initiates in the soft periodontal tissue and spreads to the underlying bone surrounding the implant, and ultimately results in bone loss. The inflammation-initiated bone loss leads to decreases in bone density and osteoblast cells, and an increase in osteoclast cells (for bone resorbing), all of which could contribute to bone resorption from the alveolar crest (under the gum tissue) down to the implant. Often recognizable by gingival inflammation and bleeding in the soft tissue around the implant, treatment for the inflammation-initiated bone loss may include cleaning the area with scalers and other methods to resolve the inflammation. Following mechanical cleaning of the soft tissue around the implant, a clot forms in this area. The goal of treatment is for the bone and soft periodontal tissue to stabilize around the implant. These treatments can be performed when minimal crestal bone loss has occurred. When more significant bone loss presents, surgical intervention is required, which includes flapping the soft tissue to expose the portion of the implant that has lost bone, cleaning that area, placing graft material, and repositioning the flap to regrow the bone. Therefore, it is also desired to have faster grafting after the procedures, so the treatment outcome could be better secured.

### SUMMARY

According to an exemplary embodiment of the present disclosure, a method for accelerating intraoral graft conversion is described. The method includes identifying a patient having bone graft material in a tooth extraction socket and one or more teeth comprising the patient's dentition, providing to the patient a vibrational dental device having a mouthpiece for contacting the dentition, and providing instructions for using the vibrational dental device. The instruction includes placing the mouthpiece over the dentition and applying a vibratory force during a predetermined number of sessions throughout a predetermined treatment period. The graft material can be converted to mature bone faster than without vibratory treatment.

According to yet another exemplary embodiment of the present disclosure, a method for accelerating graft conversion to alveolar bone is described. The method includes identifying a patient having bone graft material placed around an exposed portion of a dental implant, and one or more teeth comprising the patient's dentition, providing to the patient a vibrational dental device having a mouthpiece for contacting the dentition and/or the dental implant, and providing instructions for using the vibrational dental device. The instruction includes placing the mouthpiece over the dentition and applying a vibratory force during a predetermined number of sessions throughout a predetermined treatment period. The graft material can be converted to mature bone faster than without vibratory treatment.

Additional features and advantages of the disclosed embodiments will be set forth in part in the description that follows, and in part will be obvious from the description, or may be learned by practice of the disclosed embodiments. The features and advantages of the disclosed embodiments will be realized and attained by the elements and combinations particularly pointed out in the appended claims.

It is to be understood that both the foregoing general description and the following detailed description are examples and explanatory only and are not restrictive of the disclosed embodiments as claimed.

### BRIEF DESCRIPTION OF THE DRAWINGS

The accompanying drawings constitute a part of this specification. The drawings illustrate several embodiments of the present disclosure and, together with the description, serve to explain the principles of the disclosed embodiments as set forth in the accompanying claims. The patent or application file contains at least one drawing executed in color. Copies of this patent or patent application publication with color drawing(s) will be provided by the Office upon request and payment of the necessary fee.

The drawings are not necessarily to scale or exhaustive. Instead, emphasis is generally placed upon illustrating the principles of the inventions described herein. The accompanying drawings, which are incorporated in and constitute a part of this specification, illustrate several embodiments consistent with the disclosure and together with the description, serve to explain the principles of the disclosure. In the drawings:

FIG. 1A depicts an illustrative vibrational dental device according to one aspect of the disclosure;

FIG. 1B depicts an illustrative vibrational dental device, such as that depicted in FIG. 1A placed in the mouth of a user, according to one aspect of the disclosure;

FIG. 2A is a cone-beam computed tomography (CBCT) view of an implant placed four months post-grafting according to one example of the present disclosure;

FIG. 2B is a CBCT view of the implant of FIG. 2A following four months of integration with an exemplary use of a vibration device for five minutes daily;

FIG. 3A depicts a CBCT cross section of the maxillary molar area pretreatment demonstrating periapical pathology associated with failed endodontics;

FIG. 3B depicts a periapical radiograph demonstrating failed endodontics with associated osseous destruction;

FIG. 4A depicts a radiograph of a maxillary molar exhibiting grade 2+ mobility;

FIG. 4B depicts a radiograph of the maxillary molar of FIG. 4A following an exemplary use of a vibration device according to one aspect of the present disclosure;

FIG. 5A is an image of extraction sockets following curettage prior to socket grafting according to an example;

FIG. 5B is a radiograph of a grafted socket following extraction of bridge abutments;

FIGS. 6A-6B depict CBCT cross sections of grafted sockets four months post treatment following use of an illustrative device;

FIG. 7A is a panoramic CBCT view of an implant site demonstrating osseous graft maturation at four months according to an example;

FIG. 7B is an image of an exposed implant site showing the grafted area at four months post extraction;

FIG. 8A depicts a plan view of a dental mouthpiece according to an exemplary embodiment;

FIG. 8B is a side view of an illustrative intraoral dental device according to an exemplary embodiment;

FIG. 8C is a partial side view of an illustrative dual-arch dental device according to an exemplary embodiment;

FIG. 8D is a side view of a further illustrative intraoral dental device according to an exemplary embodiment;

FIG. 8E depicts exemplary pillar shapes according to further exemplary embodiments of the disclosure;

FIGS. 9A-9B are front and schematic cross-sectional views respectively of an upper dental arch engaging exemplary embodiments of the disclosure;

FIGS. 9C-9D are top and schematic cross-sectional views respectively of an upper dental arch engaging exemplary embodiments of the disclosure;

FIG. 10 depicts an illustrative dental device according to a further exemplary embodiment of the disclosure;

FIG. 11A is a CBCT image of a mandibular implant presenting with a radiolucent area on the mesial aspect of the implant with no clinical mobility or patient stated sensitivity;

FIG. 11B is a CBCT image of a mandibular implant following four months of daily use of the appliance showing that the radiolucency has resolved and increased osseous density is noted;

FIG. 11C is a CBCT image of a cross section before treatment demonstrating bone level on buccal/lingual of the implant and the density of the cancellous bone in contact with the implant;

FIG. 11D is a CBCT image of a cross section following treatment with the appliance demonstrating bone level on buccal/lingual of the implant and the increase in density of the cancellous bone in contact with the implant;

FIGS. 12A-B are images of a mandibular implant where the patient presented with bone loss as evidenced by decreased bone density adjacent to the implant in the absence of mobility (purple=very low density, blue=low density, green=high density, yellow=very high density);

FIGS. 12C-D are images of the mandibular implant of FIGS. 12A-B immediately following graft placement demonstrating the graft material filling the osseous void that resulted by peri-implantitis;

FIGS. 12E-F are images of the mandibular implant of FIGS. 12C-D two months post graft repair of peri-implantitis associated bone loss with daily use of low-magnitude high-frequency vibration (LMHFV) by the patient demonstrating increased density of the grafted area to blend with the native bone adjacent to it and an increase in adjacent bone density related to vibration transfer throughout the maxilla;

FIG. 13A is a chart showing comparison of PDL fibroblast between non-vibrated control and LMHFV 120 Hz over a 3-day period demonstrating statistically significant increases with the LMHFV;

FIG. 13B is a chart showing comparison of osteoblasts between non-vibrated control and LMHFV 120 Hz over a 3-day period demonstrating statistically significant increases with the LMHFV;

Reference will now be made in detail to exemplary embodiments. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

### DETAILED DESCRIPTION

It is to be understood that both the foregoing general description and the following detailed description are exemplary and explanatory only and are not restrictive of the claims.

The disclosed embodiments relate to devices, systems, and methods for accelerating graft conversion to alveolar bone. Advantageously, embodiments of the present disclosure can be implemented to convert graft material to mature bone more quickly than without. This is surprising in light of the generally held view that mechanical disruption of the graft site is detrimental to osseointegration.

When applied in immediate implant loading LMHFV can additionally advantageously accelerate bone density surrounding the implants improving the expected clinical outcome in a shorter period than traditionally observed. This is surprising in light of the generally held view that motion, including micromotion. of the implant after implantation is detrimental to the osseointegration of the implant. The application of vibration according to aspects of the current disclosure also has osseous stimulatory affects in cases where the implant will not be immediately loaded and allowed to heal before initiation of the restorative phase. Compared to without treatment, LMHFV can increase the speed and/or quality of the process of osseointegration of a bone graft including the infiltration of granulation tissue into a blood clot at an extraction site, the proliferation of epithelium into the extraction site and graft, the formation of bone spicules, and the mineralization of these spicules into mature bone. LMHFV can also increase the speed and/or quality of mineralization of immature bone spicules into mature bone.

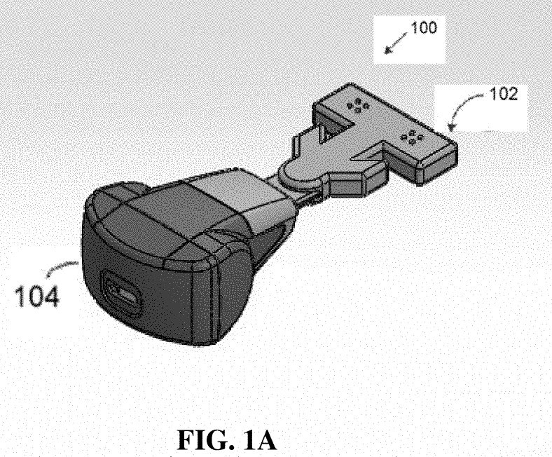

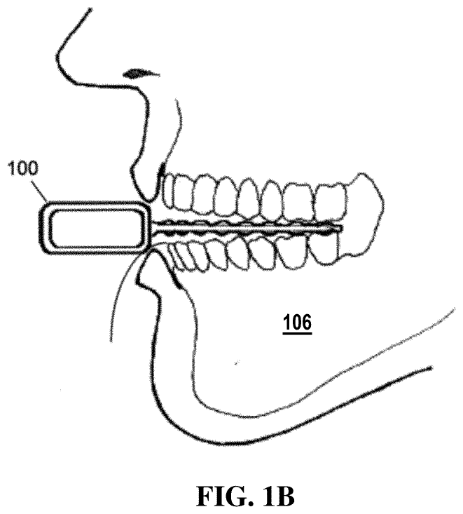

LMHFV, as indicated may be utilized immediately following implant placement when insertion torque so dictates, for example when sufficient torque to immediately load, or when clinical circumstances will not permit immediate loading, for example, insufficient insertion torque. In an aspect, with reference to FIGS. 1A and 1B, use of the device 100 providing LMHFV for 5 minutes daily accelerates osseous healing through osteogenic cell stimulation, with increased growth factor expression and angiogenesis stimulation permitting earlier loading. Further advantageously, bone density improvement is observed contributing to implant stability and better overall oral health.

LMHFV therapy according to the present disclosure is also advantageously configured to enhance and accelerate bone remodeling by improving bone density and mineral content of the bone around teeth, implants, and within grafted implant sites. In an aspect, LMHFV therapy is configured to increase bone mineral density (BMD) and improve localized osseous circulation. In an aspect, the increase in bone density improves the periodontal status of those involved teeth and contributes to a subsequent decrease in tooth mobility. This correlates to implant applications with improvement in both the BMD and circulation when utilized after implant placement or with sites that are being grafted in anticipation of later implant placement. In an aspect, LMHFV therapy advantageously contributes to the release of growth factor such as BMP2, PDGFa, and TGF β1 among others. In another aspect, LMHFV therapy is configured to increase osteoblast and PDL cell proliferation stimulation. LMHFV may also regulate gene expression-enhancing callus formation, mineralization, and remodeling of bone.

Described herein are LMHFV dental devices, which in certain embodiments include a mouthpiece configured to transmit vibration to all or a portion of the patient's teeth.

Referring to FIGS. 1A-1B, an exemplary dental device 100 includes a mouthpiece 102 operatively connected to a housing 104. The mouthpiece 102 can be separable from the housing 104 for interchangeability between users or for ease of cleaning. The mouthpiece 102 can include one or more oral tissue-contacting portion, such as a biteplate or probe for contacting teeth, gums or other oral tissues. As shown, in FIG. 1A, the mouthpiece can include a biteplate which can be appropriately shaped to cover occlusal surfaces of some or all of a patient's dentition. Other shapes for the mouthpiece are possible. For example, the mouthpiece can be configured to abut the lingual and buccal lateral sides of the alveolar ridge either with or without occlusal contact or, when no teeth are present, contact with gums overlying the alveolar ridge. A vibration generator can be located in the mouthpiece 102 or the housing 104 to vibrate the mouthpiece 102. The housing 104 can also include the electronics to run the motor the vibrator, collect usage and device operation data, collect data from sensors in the mouthpiece or base, and store data in memory. The housing 104 can include a data interface which can be wired or wireless to allow a data connection to other devices. The housing 104 can also include a power interface to allow charging of any onboard power sources, such as batteries or capacitor banks. The mouthpiece 102 can be electrically interconnected to the housing 104. FIG. 1B depicts an illustrative dental device 100, such as that described above with reference to FIG. 1A, inserted in the mouth of a human user 106 and engaging the occlusal surfaces of the molars. The mouthpiece of the dental device 100 can, as described above, be sized and shaped to contact any dental tissue, including some or all of the teeth, specific regions of the gums, or both.

As is known in the art, the vibration generator can include an electric motor connected to an eccentric weight, or can be a piezo generator, as well as other known expedients. Accordingly, when the mouthpiece 102 is placed in a patient's mouth and the dental device is 100 turned on, the vibration of the mouthpiece 102 will place vibratory force repetitively on the teeth and/or other oral tissues.

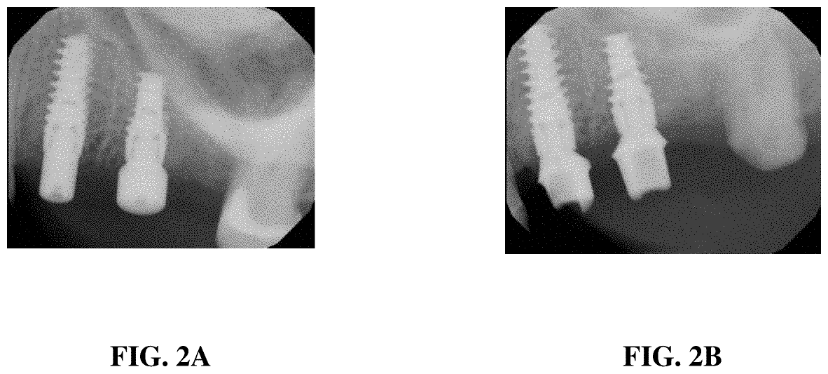

FIG. 2A is a CBCT (also referred to as C-arm CT, cone beam volume CT, flat panel CT or Digital Volume Tomography (DVT)) view showing an implant placed four months post-grafting according to an example. The implants were placed into extraction sockets into which bone had been grafted with the use of LMHV to increase bone density and integrity and to provide a more stable foundation in which to locate the implants at initial placement. Advantageously, implants such as threaded posts can be driven with a higher insertion torque than would be normally possible in type 3 or type 4 bone normally found in a long-healed posterior maxilla or 4 months post-grafting when LMHFV was not used during healing. Once loading is initiated, continued use of LMHFV will continue to further increase bone density around the implants improving their long-term prognosis through better load handling. In an example, the appliance may be used long-term as an at home therapy to preserve bone to implant contact (BIC) and potentially prevent peri-implantitis. FIG. 2B is a view of the implant in FIG. 2A following an additional four months of integration and with use of an illustrative dental device according to the present disclosure for five minutes daily. The density of the BIC during the integration period has improved, demonstrating blending of the graft with the surrounding host bone.

LMHFV also stimulates bone progenitor cells as well as increase angiogenesis resulting in acceleration of maturation of the clot in the site.

In one aspect, LMHFV has demonstrated improvement with mobility of natural teeth by its stimulation leading to improvement in the bond density making it more stable and a decrease in the PDL width.

In another aspect, as with just a clot, LMHFV offers the same effects of stimulating and accelerating conversion of the material to denser mature bone so that a dental implant may be placed into higher quality bone sooner than when LMHFV is not utilized.

In yet another aspect, LMHFV can be performed as an extraction sockets aid after a nonsurgical or surgical approach to peri-implantitis is performed. LMHFV can also be performed when any oral or facial procedure or surgery is performed and results a need for grafting, such as root canals, scaling and planning, etc. LMHFV stimulates organization of the clot to improve soft tissue reattachment, accelerates angiogenesis, and therefore improves bone formation. LMHFV also has an anti-inflammatory effect on the soft and hard tissue by accelerating and stimulating host factors to improve healing and organization, and by depressing factors that may cause inflammation. In some embodiments, accelerated healing and organization may result better pain management of the patient.

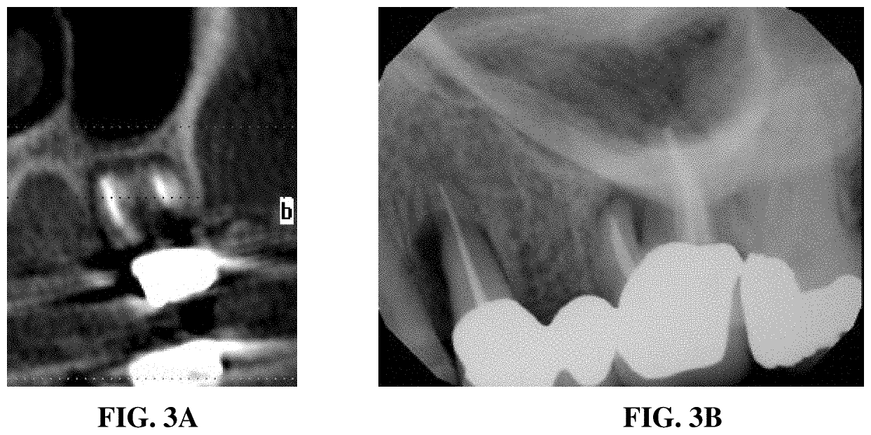

Clinically, a tooth that due to clinical issues that will not permit long-term maintenance of that tooth will be indicated for extraction. Teeth requiring extraction frequently have less dense bone surrounding them or defects related to negative biological effects such as infection. Turning to FIGS. 3A and 3B, examples of instances where bone formation is required include presentations of failed endodontics. FIG. 3A shows an image of a cross section of the maxillary molar area pretreatment demonstrating periapical pathology associated with failed endodontics that will in this instance necessitate extraction of the molar. FIG. 3B depicts a periapical radiograph demonstrating failed endodontics with associated osseous destruction that will necessitate extraction of the bridge abutment teeth.

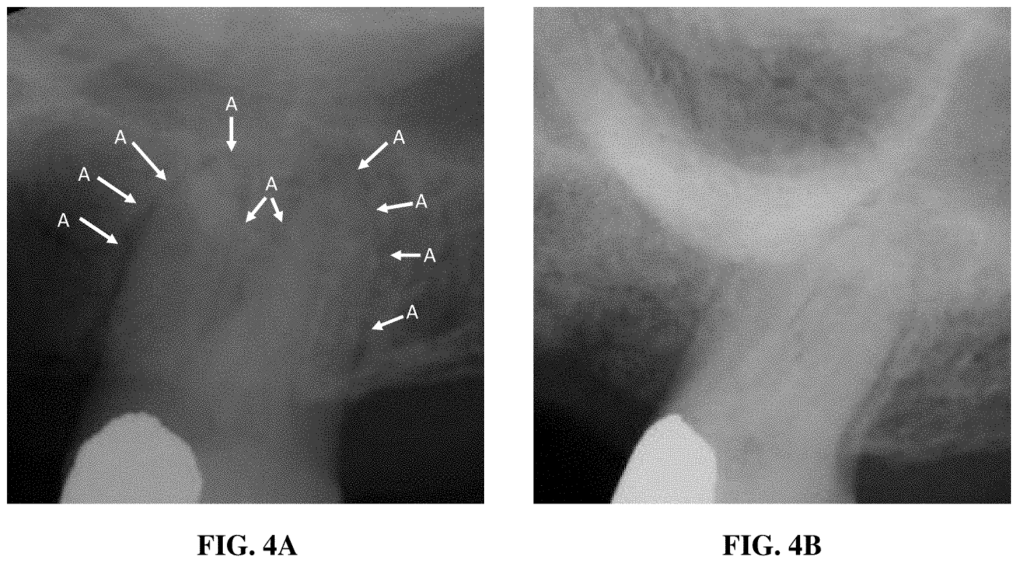

Turning to FIGS. 4A and 4B, teeth presenting with mobility will usually have a widened PDL space and lower bone density surrounding that tooth. FIG. 4A is a radiograph of a maxillary molar exhibiting grade 2+ mobility that has widened periodontal ligament space surrounding the tooth, as referenced by arrows A, and a possible lesion on the distal buccal apical area. Such widened periodontal ligament space is indicative of increased tooth mobility. FIG. 4B is a radiograph of the maxillary molar of FIG. 4A following use of the appliance for 5 minutes daily for 4 months showing that mobility has resolved and apical area has disappeared with a normal PDL space radiographically. LMHFV may be administered using illustrative device 100 (FIGS. 1A and 1B) to stimulate the bone and increase the bone density while decreasing the PDL space and associated improvement in the mobility returning to a healthy periodontal state. In an example, a patient can stimulate the bone using the appliance for five minutes daily.

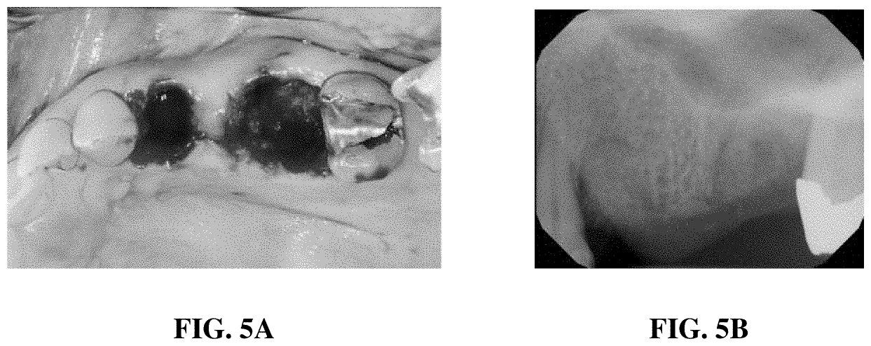

Following extraction of a problematic tooth or teeth, curettage of extraction sockets can be performed to remove any residual unhealthy or pathologic tissue. (FIG. 5A) The extraction sockets can be packed with a clinically indicated amount of an appropriate graft material and the implantation site can be closed with or without a membrane. FIG. 5B is a radiograph of a grafted socket following extraction of bridge abutments, socket curettage and socket grafting demonstrating a granular appearance of the graft material according to an example. The granular appearance of the graft material reveals a lower tissue density within the socket than the host's adjacent native bone.

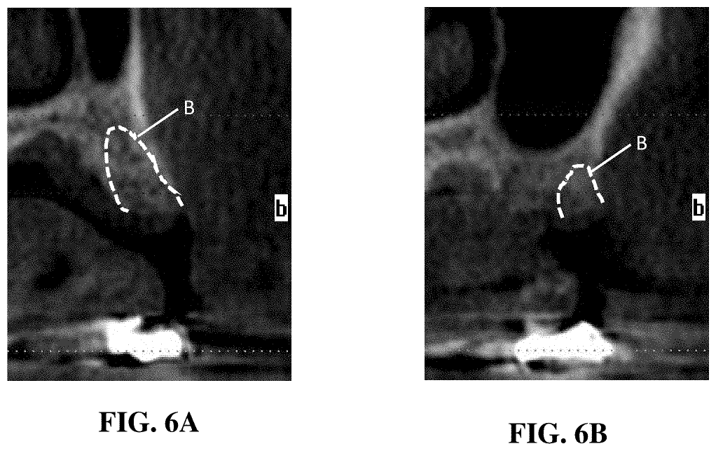

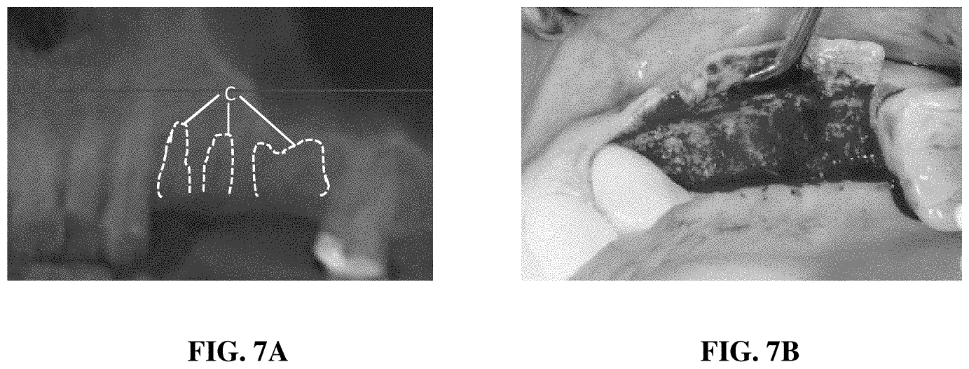

In some embodiments, the patient can be instructed to use the appliance for a prescribed time and duration to augment a grafted implant site. In an example, the patient can be instructed to use the appliance for five minutes daily over a four-month period. Turning to FIGS. 6A and 6B, when radiographically examined after a four-month healing period, the grafted site demonstrates more rapid conversion of the graft particles to blend with the surrounding host bone with similar radiographic density and trabeculation, appearing ready for implant placement. FIGS. 6A and 6B depict CBCT cross sections of grafted sockets four months post treatment following use of an illustrative device for five minutes daily demonstrating increased density of the grafted sites, which are approximately delimited by dashed lines B. FIG. 7A is a panoramic CBCT view of an implant site demonstrating osseous graft maturation at four months and ready for implant placement. Following socket grafting of the extraction sockets and use of LMHFV for 4 months, the graft sites, approximately delimited by dashed lines C, have radiographically blended with the surrounding host bone and are indistinguishable therefrom. Density improvement was accelerated with LMHV that would not be otherwise observed. FIG. 7B is an image of an exposed implant site showing the grafted area at four months post extraction and socket preservation with daily use of an exemplary device demonstrating that the osseous graft has organized to blend with the surrounding host bone.

The vibration can be applied along multiple axes or selected to be primarily on a single axis. The primary anatomic reference directions with reference to a standing human are superior-inferior (up and down), anterior-posterior (front to back), medial-lateral (side to side). Because mastication places loading on oral structures primarily in the superior-inferior direction through mandibular action, it may be advantageous to choose vibrational loading along other axes either separately or in combination.

### Vibrational Dental Devices

According to an aspect of the present disclosure, a vibrational dental device that vibrates at one or more predetermined frequencies is provided. In some embodiments the vibrational frequency is fixed within a lower bound and an upper bound. The lower bound can be greater than about 110 Hz, 105 Hz, 100 Hz, 95 Hz, 90 Hz, 85 Hz, 80 Hz, 75 Hz, 70 Hz, 65 Hz, 60 Hz, 55 Hz, 50 Hz, 45 Hz, or less. The upper bound can be greater than about 115 Hz, 120 Hz, 125 Hz, 130 Hz, 135 Hz, 140 Hz, 145 Hz, 150 Hz, or more. In some embodiments, the frequency varies within a lower and an upper bound. In some embodiments two or more frequencies, fixed or varying, are employed.

In some embodiments the duration of a treatment session can be specified to be greater than about 30 seconds, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, or more; or specified to be less than about 20 min, 19 min, 18 min, 17 min, 16 min, 15 min, 14 min, 13 min, 12 min, 10 min, 9 min, 8 min, 7 min, 6 min, 5 min, 4 min, 3 min, 2 min, 1 min, 30 seconds, or less.

FIG. 1 depicts a vibrational dental device according to an example. The vibrational dental device can include a mouthpiece and a vibrational source connected to each other. The mouthpiece is configured to be provided between the occlusal surfaces of a user's teeth, and to be bite down by the user to contact the user's dentition during the treatment. The mouthpiece can cover at least the teeth or implant around which accelerating graft conversion is desired. The vibrational source is configured to provide vibration to the mouthpiece at a preset frequency and acceleration.

To achieve the maximum desired results of accelerating graft material conversion, further studies are still needed to optimize the parameters of LMHFV. Such parameters may include frequency, acceleration, and dosage. Dosage may include duration per use, number of uses per day, or number of days of use, either consecutively or at a certain schedule.

In some embodiments, the vibrational source may be connected to the mouthpiece in such way that the vibration provided is in the sagittal plane of a user's mouth. A motor may be included in the vibrational source to provide such vibration. The motor may be of any suitable type known in the art. The motor, when in use, may be configured to provide vibration at a frequency as disclosed herein. The motor, when in use, may be further configured to provide vibration at an acceleration magnitude. In some embodiments the mouthpiece of a dental vibration device can have an acceleration within a lower bound and an upper bound. The lower bound can be greater than about 0.010 G, 0.015 G, 0.020 G, 0.025 G, 0.030 G, 0.035 G, 0.040 G, 0.045 G, 0.050 G, 0.055 G, 0.060 G, or more; or less than about 0.060 G, 0.055 G, 0.050 G, 0.045 G, 0.040 G, 0.035 G, 0.030 G, 0.025 G, 0.020 G, 0.015 G, 0.010 G, or less. The upper bound can be greater than about 0.07 G, 0.08 G, 0.09 G, 0.10 G, 0.11 G, 0.12 G, 0.13 G, 0.14 G, 0.15 G, or more; or less than about 0.15 G, 0.14 G, 0.13 G, 0.12 G, 0.11 G, 0.10 G, 0.09 G, 0.08 G, 0.07 G, or less.

The motor may be assembled into the vibrational source in an orientation that may provide vibration in such ways.

In some embodiments, sensors may be added to the vibrational dental device, either on the vibrational device, or on the mouthpiece. The sensors may be configured to detect and monitor the parameters of the vibration, for example, frequencies and acceleration magnitudes. The sensors may also be configured to detect if the user has bitten down on the mouthpiece correctly. The sensors may be accelerometers, gyroscopes, proximity sensors, pressure sensors, humidity sensors, temperature sensors, or any combinations of them.

In some embodiments, the mouthpiece could be in contact with at least the teeth or implant near which graft conversion acceleration is needed. The mouthpiece may be configured to be placed in contact with a user's dentition, between and clamped down by both occlusal surfaces of the dentition. The mouthpiece can include ridges or be without ridges. The mouthpiece can cover the entire dentition, or only a part of the dentition. The shape of the mouthpiece can be customized to cover only selected teeth or implants.

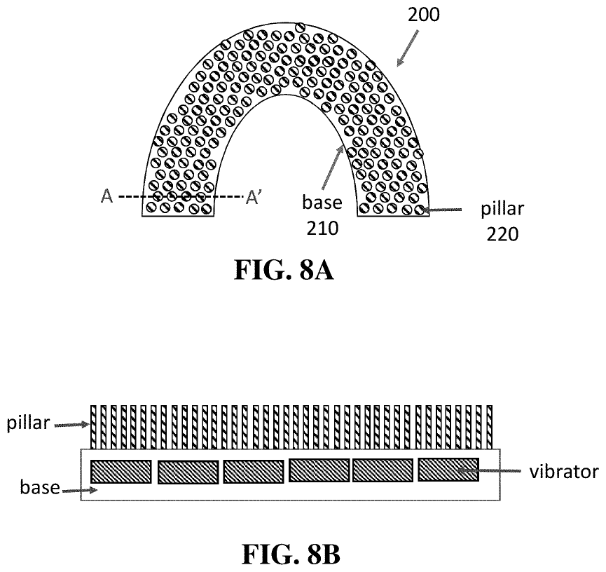

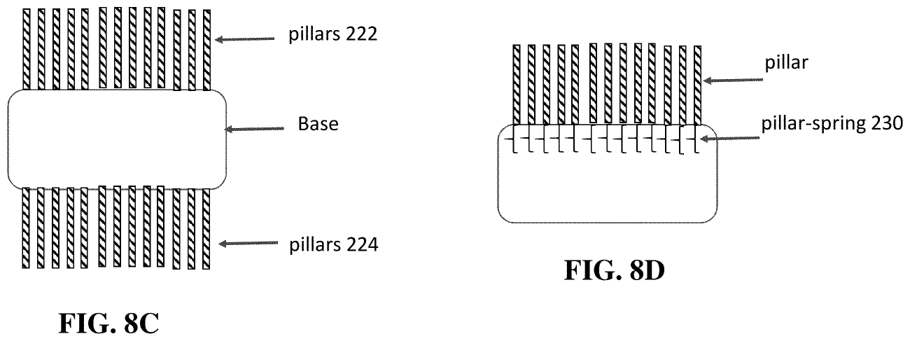

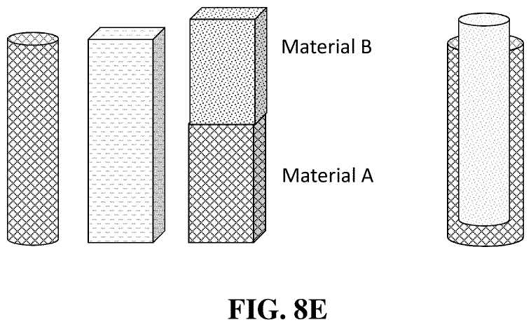

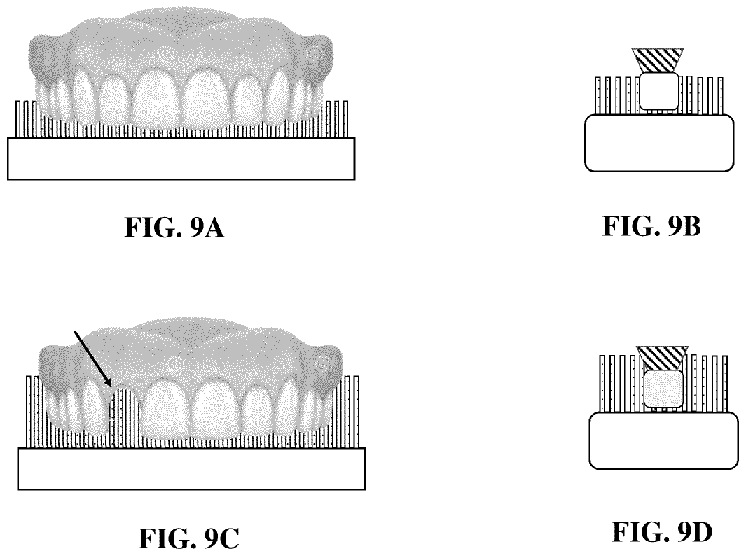

Turning to FIGS. 8A-8E, a further exemplary dental appliance 200 is depicted. The illustrative device 200 can include a base 210 and an array of bristles or pillars 220 covering the base. In an aspect, the array of pillars 220 are configured to substantially envelope one or more teeth according to an example. In some embodiments, dental appliance 200 can include a first set of pillars 222 configured to interface with a first set of teeth and a second set of pillars 224 configured to interface with a second set of teeth. In some embodiments, the array of pillars can protrude substantially parallel and vertically from the base. Subsets of pillars may also be non-parallel and apply angular stresses on the teeth. In some embodiments, each pillar can be movable with a spring 230 configured to retract when engaged with a tooth (FIG. 8D, see also FIGS. 9A-9D). FIG. 8E depicts examples of pillar shapes, having one or more materials, and configured to modify torsion on the teeth and gums and selectively enhance and/or dampen vibrations.

In some embodiments, the appliance can be configured to engage with a patient's teeth alone (FIGS. 8A-8B) or can be configured to engage with a patient's teeth and gums (FIGS. 9C-9D). As shown in FIG. 9C, the array of pillars 220, 222, 224 may gently engage with the graft site and/or the future implant site to provide stimulation to the soft tissue. Such gentle stimulation can help to increase blood flow and other cells of repair to the site, in addition to that provided by vibration conducted through neighboring teeth and tissue structures.

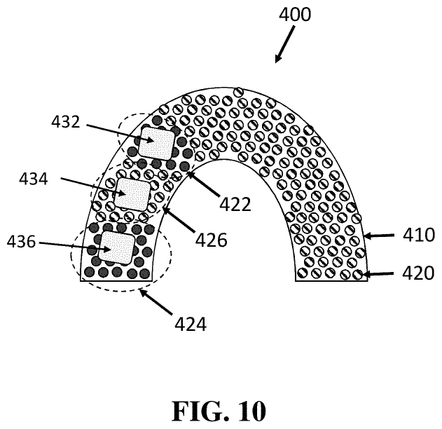

Turning to FIG. 10, in some embodiments, a granular dental appliance or appliance 400 can be configured to isolate one or more teeth or implant sites for stimulation therapy. In an example, the appliance 400 can be configured to control stimulation energy to a subset of the array of pillars. In an example, a first set of pillars 422 and a second set of pillars 424 can be configured to immobilize or isolate stimulation from at least one tooth 432 and 436 while stimulation energy is being applied to an active set of pillars 426 directed at engaging a tooth 434 or implant site.

In some embodiments, a granular dental appliance 400 can include a base portion 410 including a stimulation source such as a vibration source, a plurality of pillars 420 in communication with the base and configured to engage with at least one tooth 432, 434, and 436 and at least a portion of a gum, where a first set of pillars of the plurality of pillars is configured to immobilize or dampen vibration of at least a first tooth 432 or portion of gum, and a second set of pillars of the plurality of pillars is configured to mobilize or enhance vibration of at least a second tooth 434 or portion of gum, which can also be seen in FIGS. 9C to 9D.

According to yet another aspect of the present disclosure, a method for accelerating graft conversion to alveolar bone is described. The method including providing a vibratory dental appliance, comprising a base including a vibration source, and a plurality of pillars extending from the base and configured to engage with at least one tooth and at least a portion of a gum, determining at least one of an orientation of at least one tooth and a gum line, controlling a first vibration to a first set of pillars of the plurality of pillars, the first vibration is configured to immobilize or dampen vibration of at least a first tooth or portion of gum, and controlling a second vibration to a second set of pillars of the plurality of pillars, the second vibration is configured to mobilize or enhance vibration of at least a second tooth or portion of gum.

### Method For Accelerating Graft Material Conversion

According to yet another aspect of the present disclosure, a method for accelerating graft material conversion is described. The method includes providing the mouthpiece of the vibrational dental device to a user and providing instructions to the user. The instruction may include placement guidelines and dosage information. The dosage information may include duration of each treatment session, number of sessions in a day, number of days, etc. For example, the instruction may instruct a user to use the vibrational dental device for number of times per day. In some embodiments the treatment frequency can be specified to be once per day, twice per day, 3 times per day, 4 times per day, 5 times per day, 6 times per day, 7 times per day, 8 times per day, 9 times per day, or more. In some embodiments the duration of treatment can be specified to be about 1 day, 1 week, 2 weeks, 3 weeks, 1 month, 2 months, 3 months, 4 months, 5 months, 6 months, 7 months, 8 months, 9 months, 10 months, 11 months, 1 year, or more.

In some embodiments, the method may further include configuring the vibrational source providing an axial vibratory force to the mouthpiece. The axial vibratory force may be eventually applied to the dentition through the mouthpiece, which is clamped down by the teeth. The vibratory force (e.g., acceleration magnitudes, frequencies, etc.) can be adjusted by selecting preset values, or fine-tuned by users, technicians, or healthcare professionals.

According to yet another aspect of the present disclosure, a method for detecting graft material conversion is described. The method includes steps of identifying an implant site, providing a graft at the implant site, applying a stimulus to a portion of the implant site, sensing a baseline response at the implant site, applying one or more vibration sessions over a period of time, sensing at least one second response at the implant site, and determining a osseous status based on a comparison between the baseline response and one or more second responses. In some embodiments, the method may further include implanting a dental implant at the implant site based on the osseous status. In some embodiments, the method may further include applying a stimulus to a portion of the implant site with the dental implant.

In some embodiments, the stimulus applied can be one or electrical energy, light energy, and a mechanical dynamic load that is either isotonic or isometric. In addition, the stimulus can be applied to a portion of the implant site symmetrically or asymmetrically on one side of the implant site or across the implant site such as across a facial side and lingual side or mesial side and distal side (see FIG. 3A). In some embodiments, sensing a baseline response can include information informing an osseous density at the implant site.

### Examples

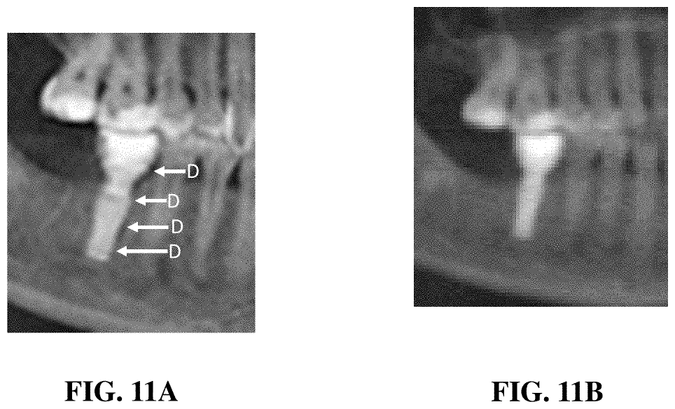

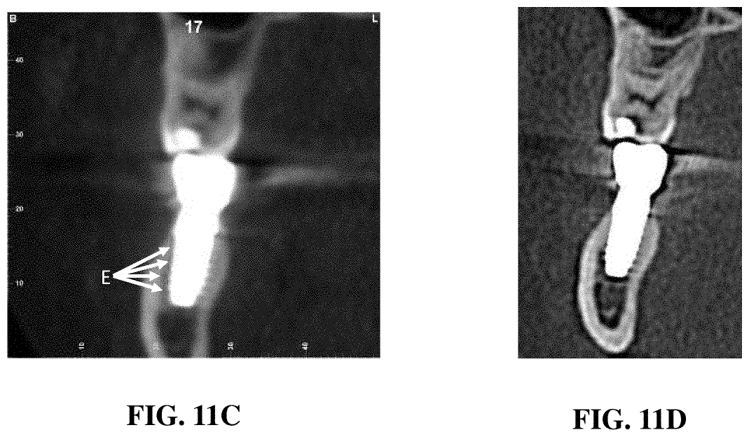

FIG. 11A is a CBCT image of a mandibular implant presenting with a radiolucent area on the mesial aspect of the implant with no clinical mobility or patient stated sensitivity. Radiographic evidence of a space present between the implant and bone on the mesial, indicated by arrows D, indicate peri-implantitis. LMHFV was utilized by the patient daily, increasing bone density and eliminating the mesial space and the peri-implantits. The patient reported no pain or mobility during or following treatment. FIG. 11B is a CBCT image of a mandibular implant following four months of daily use of the appliance showing that the radiolucency has resolved and increased osseous density is noted. The implant was rescued without the need for surgical intervention.

FIG. 11C is a CBCT image of a cross section before treatment demonstrating bone level on buccal/lingual of the implant and the density of the cancellous bone in contact with the implant. This posterior mandibular implant presented with radiographic evidence of a space present between the implant and bone on the mesial, as indicated by arrows E, indicating peri-implantitis with no mobility of the implant or pain noted by the patient. FIG. 11D is a CBCT image of a cross section following daily LMHFV treatment with the appliance demonstrating bone level on buccal/lingual of the implant and the increase in density of the cancellous bone in contact with the implant. As a result of the treatment, the patient eliminated the peri-implantitis and rescued the implant without the need for surgical intervention.

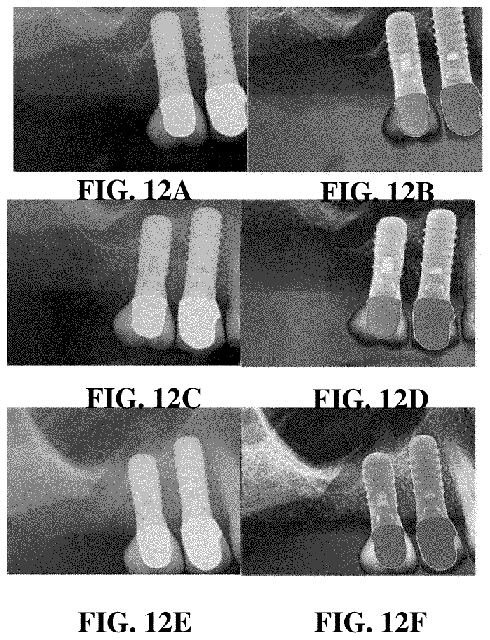

FIGS. 12A (unretouched) and 12B (colorized) are radiographic images of a mandibular implant where the patient presented with bone loss as evidenced by decreased bone density adjacent to the implant in the absence of mobility (purple=very low density, blue=low density, green=high density, yellow=very high density). The patient was subject to surgical intervention to debride the area and clean the exposed threads plus place osseous graft material to fill in the defect caused by the inflammation associated with the peri-implantitis. FIGS. 12C and 12D are images of the mandibular implant of FIGS. 12A and 12B immediately following graft placement demonstrating the graft material filling the osseous void that resulted by peri-implantitis. FIGS. 12E-F are images of the mandibular implant of FIGS. 12C and 12D two months post-graft repair of peri-implantitis associated bone loss with daily use of LMHFV by the patient demonstrating a disappearance of peri-implantitis, increased density of the grafted area to blend with the native bone adjacent to it and an increase in adjacent bone density related to vibration transfer throughout the maxilla. Two months post treatment after daily LMHFV use, and improvements is seen in the affect (grafted) implant, as well as high density bone along the entire length. Additionally, comparing distant bone (to the left of the implants) were no teeth or implants were present initially, the density is of type 4 quality typically found in the posterior maxilla. Following LMHFV and its transmission throughout the bone, a distance from the implants being treated, a significant increase in bone density raising that bone to at least type 2, demonstrating LMHFV transmission greatly improved bone quality at and adjacent to the area being treated. Bone density improvement to this degree is not observed with graft placement without the use of LMHFV.

The distal maxillary implant presented with 50% bone loss and very low bone density surrounding the implant, indicative of peri-implantitis. When compared to the mesial implant that had no bone loss, a significant deterioration on the affected implant is evident. The patient utilized LMHFV daily for 5 minutes and the radiograph taken at 2 months demonstrates successful graft integration into native bone and a rescue of the implant.

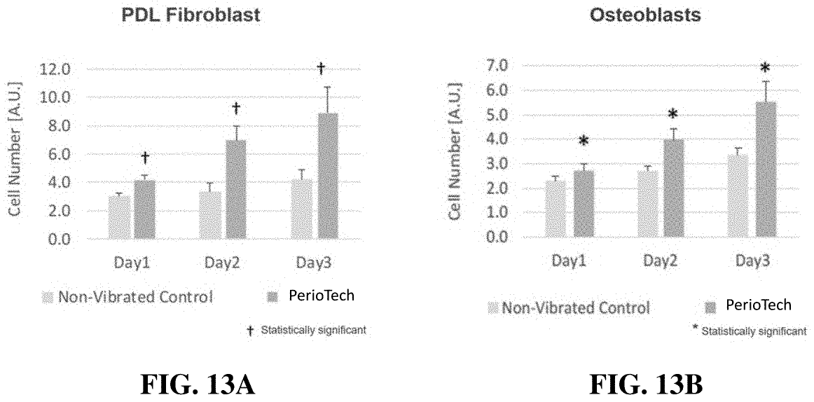

FIG. 13A is a chart showing comparison of PDL fibroblast between non-vibrated control and LMHFV 120 Hz over a 3-day period demonstrating statistically significant increases with the LMHFV. FIG. 13B is a chart showing comparison of osteoblasts between non-vibrated control and LMHFV 120 Hz over a 3-day period demonstrating statistically significant increases with the LMHFV.

The foregoing descriptions have been presented for purposes of illustration. They are not exhaustive and are not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

It should be noted that, the relational terms herein such as “first” and “second” are used only to differentiate an entity or operation from another entity or operation, and do not require or imply any actual relationship or sequence between these entities or operations. Moreover, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the terms “and/or” and “or” encompass all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

It is appreciated that the above-described embodiments can be implemented by hardware, or software (program codes), or a combination of hardware and software. If implemented by software, it may be stored in the above-described computer-readable media. The software, when executed by the processor can perform the disclosed methods. The computing units and other functional units described in this disclosure can be implemented by hardware, or software, or a combination of hardware and software. One of ordinary skill in the art will also understand that multiple ones of the above-described modules/units may be combined as one module/unit, and each of the above-described modules/units may be further divided into a plurality of sub-modules/sub-units.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the disclosure disclosed herein. It is intended that the specification and examples be considered as exemplary only, with a true scope and spirit of the disclosure being indicated by the following claims. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A method for accelerating graft conversion to alveolar bone, comprising:
identifying a patient having bone graft material in a tooth extraction socket and one or more teeth comprising the patient's dentition;
providing to the patient a vibrational dental device having a mouthpiece for contacting the dentition; and
providing instructions for using the vibrational dental device, the instruction comprising:
placing the mouthpiece over the dentition;
applying a vibratory force during a predetermined number of sessions throughout a predetermined treatment period;
wherein the graft material is converted to mature bone faster than without vibratory treatment.

2. The method of claim 1, wherein the frequency is ranging from 45 Hz to 150 Hz.

3. The method of claim 1, wherein the duration is ranging from 30 seconds to 20 minutes.

4. The method of claim 1, wherein the acceleration of the mouthpiece ranged from 0.010 G to 0.15 G.

5. The method of claim 2, wherein the frequency is about 113 Hz.

6. The method of claim 1, wherein the infiltration of granulation tissue into the clot is enhanced compared to than without vibratory treatment.

7. The method of claim 1, wherein the proliferation by epithelium into the extraction site into the is accelerated compared to than without vibratory treatment.

8. The method of claim 1, wherein formation of bone spicules is accelerated in the region of the socket compared to than without vibratory treatment.

9. The method of claim 1, wherein formation of bone spicules is increased in the region of the socket compared to than without vibratory treatment.

10. The method of claim 1, wherein mineralization is accelerated in the region of the socket compared to than without vibratory treatment.

11. The method of claim 1, wherein mineralization is increased in the region of the socket compared to than without vibratory treatment.

12. The method of claim 1, wherein alveolar space is decreased in the region of the socket compared to than without vibratory treatment.

13. The method of claim 1, wherein the session time is from 30 seconds to 20 minutes.

14. The method of claim 1 where sessions are repeated daily/every other day/semi-weekly/weekly.

15. The method of claim 1, wherein the treatment period is from 1 day to 1 year.

16. The method of claim 1, further comprising:
determining if the actual frequency or acceleration is above or below the set frequency or the set acceleration while the mouthpiece is vibrated against the occlusal surfaces of the patient's teeth; and
adjusting the actual frequency or actual acceleration based upon the determination.

17. The method of claim 1, wherein the graft material includes demineralized cancellous allograft.

18. The method of claim 1, wherein the graft material includes autologous L-PRF.

19. A method for accelerating graft conversion to alveolar bone, comprising:
identifying a patient having bone graft material placed around an exposed portion of a dental implant, and one or more teeth comprising the patient's dentition;
providing to the patient a vibrational dental device having a mouthpiece for contacting the dentition and/or the dental implant; and
providing instructions for using the vibrational dental device, the instruction comprising:
placing the mouthpiece over the dentition;
applying a vibratory force during a predetermined number of sessions throughout a predetermined treatment period;
wherein the graft material is converted to mature bone faster than without vibratory treatment.
